(19) United States
(12) Patent Application Publication
(10) Pub. No.: US 2003/0131853 A1
(43) Pub. Date: Jul. 17, 2003

(54) Oro-pharyngeal airway and gas-assisted injection molding method therefor

(76) Inventors: William H. Wall JR., Norcross, GA (US); William H. Wall III, Norcross, GA (US)

Correspondence Address: Eric P. Schellin, Suite 704, 2121 Crystal Drive, Arlington, VA, 22202, US

(21) Appl. No.: 10/046,767

(22) Filed: Jan. 17, 2002

(51) Int. Cl. A61M 16/00

(52) U.S. Cl. 128/207.14; 128/200.26

(57) ABSTRACT

An oro-pharyngeal apparatus defining both a suction conduit adapted to receive an operative connection to a suction pump for removing liquids from a patient's throat and at least one airway through which the patient can breath during medical or dental treatment. The invention allows simultaneous use of the suction conduit and airway. Alternately, an oxygen pump can be connected to the conduit for insufflation of the patient. The airway is provided with a color coded ring to indicate a pre-determined size of the airway to accommodate various sized individuals. The device is fabricated by gas assisted injection molding techniques.

### FIELD OF THE INVENTION

This invention relates to an oro-pharyngeal airway and more specifically provides an airway operable to permit fluids, mucous, blood or vomitus material to be removed from the pharynx without the removal of the airway from the patient's mouth, to permit insufflation of the patient's lungs and to permit normal breathing on the part of the patient while the airway is in place.

### BACKGROUND

The prior art includes a number of airway designs operable for providing an airway into a patient's throat area to permit air to enter and escape during normal breathing. However, during certain medical conditions, such as following general anesthesia, comatose, emergency conditions or in newborns, it is necessary to develop suction in the patient's throat to remove fluids, mucous, blood or vomitus material. In using most of the prior art airways, the airway must be removed and a catheter tube inserted in the throat area to perform the suction operation. Also, most prior art airways must be removed before insufflation of a patient can be effected, unless an oxygen mask or nasal oxygen tube is used. Although certain prior art airways provide a passageway for insertion of a suction tube to evacuate the throat or pharynx almost none has provided an integral suction passageway for connection to an external suction apparatus or oxygen supply.

### SUMMARY OF THE INVENTION

Briefly, the present invention comprises a suction airway defining a breathing passageway as well as conduit for connection to either a suction means or an oxygen supply means which can be easily attached to the conduit without removing the airway.

More particularly, the invention pertains to an oropharyngeal suction airway comprising an elongate body adapted to extend from a forward end thereof adapted to be engaged by a person's mouth to a rearward end thereof adjacent to the person's pharynx. The body includes a partially enclosed elongate conduit means extending along the length of the body and open at the forward or proximate end and rearward or distal end thereof. Means define an elongate airway for breathing by the person, adjacent to the conduit means, said means is located at the proximal end of the conduit means for receiving an operative connection to a pump means without obstructing the flow of air through the airway means.

In one aspect, the airway means is defined by a pair of parallel displaced ribbon-like flanges, the flanges being fixed to opposite sides of the elongate conduit means.

In another aspect the proximal end of the conduit means defines a radial suction control opening therein.

In a preferred embodiment the body further includes a pair of parallel ribbon-like flanges defining a straight proximal section and an arcuate distal section; the flanges being held in spaced apart relation to each other by a relatively rigid conduit integrally attached to the flanges. The flanges define radially extending flanges as lip elements at the proximal end thereof for engaging the outside of a person's mouth, one of the lip elements defining a radial suction control opening therein in communication with the interior of the rigid conduit. The rigid conduit defines at its proximal end a tapered end frictionally receiving a tube connected to a pump means.

The airway of the invention will function as an airway during certain conditions, and will function as a combination airway and pharynx evacuator when necessary.

The invention also provides a pharynx evacuation system which can be used in combination with a standard anesthesia mask and connection means and can be used with patient insufflation systems.

The device of the present invention is supplied with a side hole means near the distal end of the conduit. The hole is perpendicular to the conduit.

The device of the present invention is supplied with a colored ring that is mounted radially between the end of a curved lip retaining flange and the end of the tapered portion of the proximal end of the device. This colored ring is designed to color code the various sizes of the airway devices of the present invention whereby the care giver attendant can quickly select the appropriate sized airway device by looking for the appropriately colored ring on the airway device.

Additionally, while airway device of the invention have been fabricated as by injection molding the present devices have been fabricated as by gas assisted injection molding thereby giving excellent definition.

These and other aspects and advantages of the details of construction will become apparent after reading the following description of the illustrative embodiment, with reference to the attached drawings.

### BRIEF DESCRIPTION OF THE DRAWINGS

FIG. 1 is a side elevational view taken longitudinally along a transparent oropharyngeal suction airway embodying the present invention.

FIG. 2 is a cross sectional view taken along lines 2-2 of FIG. 1.

FIG. 3 is an elevational view similar to that of FIG. 1 but shows the colored size identifying ring exploded from its position.

FIG. 4 is a cross sectional view taken along lines 4-4 of FIG. 3.

FIG. 5 is a perspective view of the device of the present as viewed from its proximal end towards its distal end.

FIG. 6 is similar to the view of FIG. 5 but shows the colored identifying ring separate from the device.

FIG. 7 is a perspective view of the device rotated 90° as compared to FIG. 5.

FIG. 8 is similar to FIG. 7 but shows the colored identifying ring separate from the device.

### DETAILED DESCRIPTION

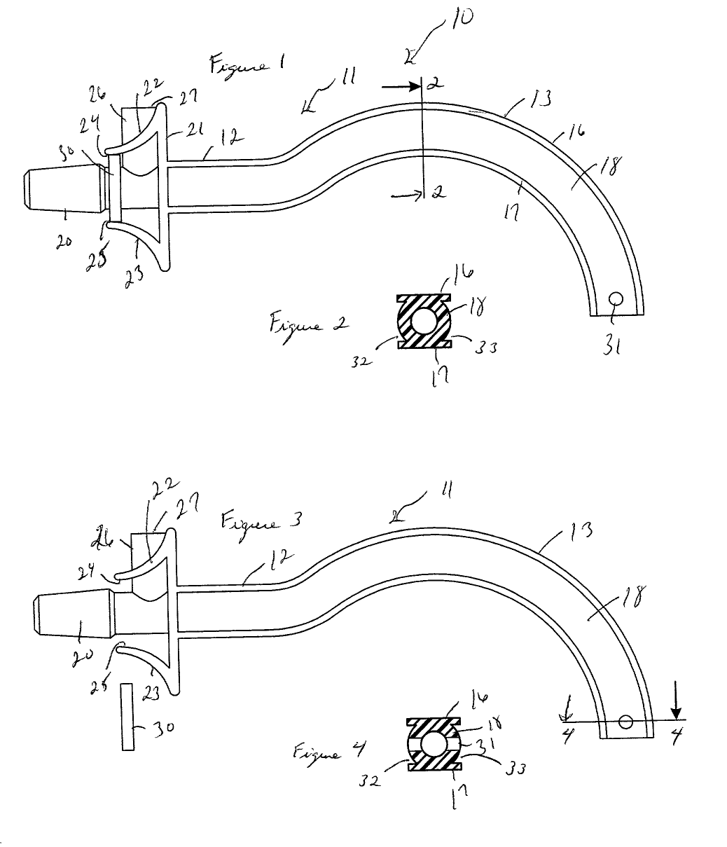

Referring now in more detail to the drawings, in which like numerals represent like parts throughout the several views, FIG. 1 depicts a side elevational view of an oro-pharyngeal suction airway 10 embodying the invention. The suction airway 10 includes an elongate body 11 formed of a suitable rigid material, such as a relatively lightweight thermoplastic that can be gas assisted injection molded into the detailed shape. The gas assisted injection method is in any conventional known method which has become important in the injection molding art because of its well known merit.

The body 11 includes a proximal straight section 12 and a distal arcuate section 13 detailed by a pair of spaced longitudinally extending parallel ribbon-like flange elements 16 and 17. They are preferably formed integrally with conduit 18.

The airway 10 is preferably fabricated from polyethylene material in a known gas assisted injection molding process employing a mold. The polyethylene material is pressurized towards a cavity in a mold. The mold is also supplied through a nozzle and conduit therefor with a supply of nitrogen gas or other suitable gas at high pressure from a suitable container.

The method for manufacturing the airway is initiated by simultaneously feeding or injecting the polymer material and the nitrogen gas into the cavity of the mold whereby the nitrogen gas pushes the polymer material against the cavity walls of the mold, so that the material forms the airway body of the present invention. This gas-assisted injection molding is already known and is therefore not described in detail here. An example of the fabrication of an analogous suction device employing a gas assisted injection molding technique can be seen in U.S. Pat. No. 5,803,731 to Nordstroem. Other patents depicting gas assisted injection molding are U.S. Nos. 4,948,547 to Hendry; 5,028,377 to Hendry; 5,039,463 to Loren, 5,208,046 to Shah et al; 5,284,429 to Schneider et al and 5,295,800 to Nelson. All of these patents are incorporated herein in their entireties by reference.

The proximal straight section 12 terminates in a forwardly extending tapered portion 20, to which a flexible hose can be attached (not shown). Between the tapered portion 20 and the termination of the two flanges 16 and 17 is a radially extending flange 21 which extends in two directions. Both the top edge and the bottom edge of the flange 21 have arcuate walls 22 and 23 sloping towards each other. The walls 22 and 23 extend in an arcuate manner longitudinally towards the conduit 18 but just shy of the widest portion of the tapered portion 20. The confronting free edges 24 and 25, respectively, of arcuate walls 22 and 23, terminate in a spaced relationship from conduit 18.

The conduit 18 has a T-connection 26 which passes through upper arcuate wall 22. The T-connection 26 terminates in an upwardly facing orifice 27. The orifice 27 can be valve closed as by a thumb or the like.

A feature of the present invention is the use of a ring 30 positioned around conduit 18 and in juxtaposition beneath the edges 24 and 25 of arcuate walls 22 and 23, respectively. The ring 30 is desired to be colored whereby it can identify the size of the airway. It will be well appreciated that different sized airways will be necessary to accommodate individuals of different dimensions especially when the airways of the present invention are to be employed by children of varying ages. The use of colored rings is used to color code different sizes of airways.

The airway of the present invention has an orifice 31 near the distal most end of the airway and conduit 18. The orifice ensues the side-ways gas introduction into the patient even when the distal most end of the conduit 18 is clogged.

In FIG. 4 it will be seen that the side orifice 31 is on opposite sides of the conduit 18.

Both FIGS. 2 and 4 clearly depict the flanges 16 and 17 with the conduit 18 sandwiched therebetween.

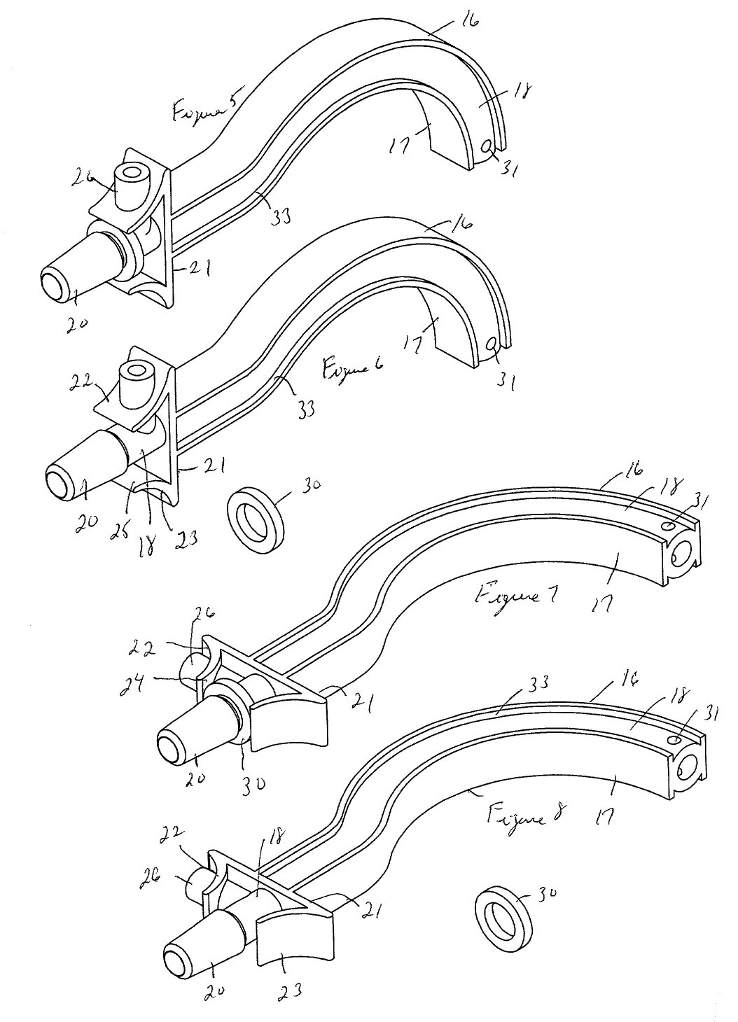

FIGS. 5 and 6 is a perspective view of the airway 11 where the airway is in a standing position.

FIGS. 7 and 8 is a perspective view of the airway 11 with the airway shown as lying on its side.

In FIGS. 6 and 8 the colored size rings 30 are depicted as being removed to thereby clearly show the airway without the size indicia rings.

It will be seen that the suctioning or aerating capability of the suction airway is improved with a plurality of openings 31 which are made radially into the distal portion of the tube 18.

In operation of the oral-pharyngeal suction airway 11, the device is inserted into the patient's mouth until the curved distal section 13 extends into the back of the patient's throat adjacent to the pharynx. The back side of the flange 21 is placed against the outer surface of the patient's lips and the patient is instructed to bite down on the proximal straight portion 12 of flanges 16 and 17. At this juncture, the patient is able to breathe through the channels 32 and 33 on the sides of conduit 18 and between flanges 16 and 17. If no other apparatus is connected to the tapered portion 20, then the patient can also breathe through the conduit 18. In the placement of the suction airway 11, the attending person can grasp the arcuate portions 22 and 23 with the fingers in order to control the location of the suction airway 11.

When it is necessary to perform a throat evacuation to remove fluid, mucous, blood or vomitus material from the throat, a flexible tubular conduit (not shown) is frictionally engaged over the projection tapered portion 20. A suction pump apparatus also connected to the flexible tube is operated to create suction within the tube 18 to withdraw such fluids from the patient's throat. This can be performed without removing the suction airway 11 from the patient's mouth. Alternately, insufflation of the patient's lungs can be accomplished by connecting the tube to an oxygen supply apparatus in order to pump a stream of oxygen out of the end of the conduit 18 down the patient's throat to the patient's lungs. During either suction or oxygen supply operations, the patient is still able to breath through the channels 32 and 33. The vent opening 27 can be used as a valve to control the strength of suction by means of the attending person's fingertip closing off all or a portion of the opening 27. If continuous full strength suction is desired, the vent opening 27 can be plugged with a rubber stopper (not shown) or the like.

While this invention has been described in detail, it will be understood that variations and modifications can be effected within the spirit and scope of the invention as described hereinbefore and as defined in the appended claims.

What is claimed is:

1. An oro-pharyngeal suction airway comprising an elongate body adapted to extend from a proximal end thereof adapted to be engaged by a person's mouth to a distal end thereof adjacent to the person's pharynx, said body including:
enclosed elongate conduit means extending along the length of said body and being open at the proximal and distal ends thereof;
means located at the proximal end of said conduit means for receiving an operative connection to a pump means without obstructing the flow of air through said airway means;
said airway means being defined by a first elongate ribbon-like flange on along the top of said elongate conduit and a second elongate ribbon-like flange at along the bottom of said elongate conduit;
each of said flanges having a width larger than the outer diameter of said elongate conduit whereby said flanges and said elongate conduit together define an elongate airway for breathing by said person when employed;
said airway being constructed of a thermoplastic polymer and said airway is formed in a gas assisted injection mold.

2. The apparatus of claim 1, wherein said for receiving an operative connection to a pump means comprises a tapered conduit end shaped to receive and frictionally retain a flexible conduit connected to said pump means.

3. The apparatus of claim 2 wherein the said first and second elongate flanges terminate proximally at a first side of an abutment flange which is radial to said elongate conduit and is spaced from said tapered conduit, said abutment flange having at second side, a first and a second relatively short longitudinally extending plates on one side of the elongate conduit and the second on the other side of the elongate conduit; a ring radially mounted about said elongate conduit, said ring having an outer diameter whereby it is in frictional engagement between an underside of each of said extending plates, said ring being in contrasting color with respect to said airway whereby to denote a preselected identifying size.

4. The apparatus of claim 3 wherein said pump means comprises a suction pump.

5. The apparatus of claim 3 wherein said pump means comprises an oxygen supply pump.
